(19) United States
(12) Patent Application Publication
(10) Pub. No.: US 2024/0173384 A1
(43) Pub. Date: May 30, 2024

(54) TREATMENT OF PEDIATRIC TYPE 2 DIABETES MELLITUS PATIENTS

(71) Applicant: Sanofi-Aventis Deutschland GmbH, Frankfurt (DE)

(72) Inventors: Karin Bergmann, Frankfurt am Main (DE); Agnes Hincelin-Mery, Paris (FR); Christelle Jan, Paris (FR)

(21) Appl. No.: 18/366,130

(22) Filed: Aug. 7, 2023

Related U.S. Application Data
(63) Continuation of application No. 17/028,400, filed on Sep. 22, 2020, now abandoned.
Application No. 17/028,400 is a continuation of application No. 16/266,873, filed on Feb. 4, 2019, now abandoned.
Application No. 16/266,873 is a continuation of application No. 14/995,910, filed on Jan. 14, 2016, now abandoned.

(30) Foreign Application Priority Data
Jan. 16, 2015 (EP) 15151488.2

(51) Int. Cl. A61K 38/26; A61K 31/155; A61K 38/28

(52) U.S. Cl. CPC A61K 38/26; A61K 31/155; A61K 38/28

(57) ABSTRACT

The present invention refers to lixisenatide for use in pediatrics.

### RELATED APPLICATIONS

This application is a continuation of U.S. patent application Ser. No. 16/266,873, filed Feb. 4, 2019, which is a continuation of U.S. patent application Ser. No. 14/995,910, filed Jan. 14, 2016, which claims the benefit of European Patent Application No. 15151488.2, filed Jan. 16, 2015. The contents of the aforementioned applications are hereby incorporated by reference in their entirety.

### DESCRIPTION

Subject of the present invention is desPro36Exendin-4(1-39)-Lys6-NH2 (AVE0010, lixisenatide) or/and a pharmaceutically acceptable salt thereof, for use in pediatrics. Yet another subject of the present invention is a pharmaceutical combination for use in pediatrics, said combination comprising (a) lixisenatide or/and a pharmaceutically acceptable salt thereof, and (b) metformin or/and a pharmaceutically acceptable salt thereof.

Yet another aspect is a method for treatment of a pediatric patient, said method comprising administering lixisenatide or/and a pharmaceutically acceptable salt thereof, optionally in combination with metformin, to a pediatric patient in need thereof.

In a healthy person the release of insulin by the pancreas is strictly coupled to the concentration of blood glucose. An increased level of blood glucose, as appears after meals, is rapidly counterbalanced by a respective increase in insulin secretion. In fasting condition the plasma insulin level drops to a basal value which is sufficient to ensure the continuous supply of glucose to insulin-sensitive organs and tissues and to keep the hepatic glucose production at a low level at night.

In contrast to type 1 diabetes, there is not generally a lack of insulin in type 2 diabetes mellitus but in many cases, particularly in progressive cases, the treatment with insulin is regarded as the most suitable therapy, if required in combination with orally administered anti-diabetic drugs. An increased glucose level in the blood over several years without initial symptoms represents a significant health risk. It could clearly be shown by the large-scale DCCT study in the USA (The Diabetes Control and Complications Trial Research Group (1993) N. Engl. J. Med. 329, 977-986) that chronically increased levels of blood glucose are a main reason for the development of diabetes complications. Examples for diabetes complications are micro and macrovascular damages that possibly manifest themselves in retinopathies, nephropathies or neuropathies and lead to blindness, renal failure and the loss of extremities and are accompanied by an increased risk of cardiovascular diseases. It can thus be concluded that an improved therapy of diabetes primarily has to aim keeping blood glucose in the physiological range as closely as possible.

A particular risk exists for overweight patients suffering from type 2 diabetes mellitus, e.g. patients with a body mass index (BMI)≥30 kg/m². In these patients the risks of diabetes overlap with the risks of overweight, leading e.g. to an increase of cardiovascular diseases compared to type 2 diabetes mellitus patients being of a normal weight.

Until recently, type 2 diabetes mellitus (T2DM) was almost exclusively an adult disease. Coinciding with the increasing prevalence of obesity in children, the incidence of T2DM in children and adolescents has markedly increased to the point that it accounts for as many as one third of all the new cases of T2DM diagnosed in adolescent.

Children/adolescents with T2DM are usually diagnosed over the age of 10 years, in middle to late puberty, when due to physiological changes in the GH/IGF-1 axis insulin resistance develops. Like in adults, the incidence of type 2 diabetes in children/adolescents is highest in some ethnic populations (e.g. American Indians, African American, Asian/pacific Islander and Hispanics) (Canadian Diabetes Association Clinical Practice Guidelines Expert Committee, Type 2 Diabetes in Children and Adolescents, 2008 Clinical Practice Guidelines, S162-S167).

Diabetes is a therapeutic area for which the EMA Paediatric Working Party considers that research and development of medicinal products for children should be performed. Type 2 diabetes may have an earlier and more aggressive course in pediatric patients; therefore, they are likely to be at higher risk for developing complications and need the best possible glycemic control in the early stage of their disease.

T2DM in children differs from adults in a number of ways that have an important impact on potential treatment. Puberty appears to play a major role in the development of type 2 diabetes in children. During puberty, there is increased resistance to the action of insulin, resulting in hyperinsulinemia. Growth hormones have been considered as candidates for causing insulin resistance during puberty. The mean growth hormone levels increase transiently during puberty coincidental with the decrease in insulin action. Given this information, it is not surprising that the peak age at presentation of type 2 diabetes in children coincides with the usual age of mid-puberty. In an individual who has a genetic predisposition for insulin resistance, compounded with environmental risk exposure, the additional burden of insulin resistance during puberty may tip the balance from a state of compensated hyperinsulinemia with normal glucose tolerance to inadequate insulin secretion and glucose intolerance that continues beyond puberty (American Diabetes Association, Diabetes Care 2000, 23(3): 381-389).

In the US, about 12% of type 2 pediatric diabetes mellitus patients received metformin monotherapy and about 34% received insulin monotherapy (Dombrowsky and Barrett, Type II Diabetes Mellitus in Children: Analysis of Prevalence Based on the Pediatric Heath Information System (PHIS) Database, American College of Clinical Pharmacology Annual Meeting Sep. 22-24, 2013, Bethesda, Maryland). In many pediatric type 2 diabetes mellitus patients, progression of the disease is rapid, and control of hyperglycemia may become insufficient even at maximal tolerated doses of metformin.

However there are no reported studies describing the efficacy of GLP-1 analogs in the pediatric population.

Metformin is a biguanide hypoglycemic agent used in the treatment of non-insulin-dependent diabetes mellitus (type 2 diabetes mellitus) not responding to dietary modification. Metformin improves glycemic control by improving insulin sensitivity and decreasing intestinal absorption of glucose. Metformin is usually administered orally. However, control of type 2 diabetes mellitus in obese patients by metformin may be insufficient. Thus, in these patients, additional measures for controlling type 2 diabetes mellitus may be required.

Metformin is the international nonproprietary name of 1,1-dimethylbiguanide (CAS number 657-24-9).

The compound desPro36Exendin-4(1-39)-Lys6-NH2 (AVE0010, lixisenatide) is a derivative of Exendin-4. AVE0010 is disclosed as SEQ ID NO:93 in WO 01/04156:

SEQ ID NO: 1: lixisenatide (44 amino acids)
H-G-E-G-T-F-T-S-D-L-S-K-Q-M-E-E-E-A-V-R-L-F-
I-E-W-L-K-N-G-G-P-S-S-G-A-P-P-S-K-K-K-K-K-
K-NH₂
SEQ ID NO: 2: exendin-4 (39 amino acids)
H-G-E-G-T-F-T-S-D-L-S-K-Q-M-E-E-E-A-V-R-L-F-
I-E-W-L-K-N-G-G-P-S-S-G-A-P-P-P-S-NH₂

Exendins are a group of peptides which can lower blood glucose concentration. The Exendin analogue lixisenatide is characterised by C-terminal truncation of the native Exendin-4 sequence. Lixisenatide comprises six C-terminal lysine residues not present in Exendin-4.

Lixisenatide is also termed des-38-proline-exendin-4(Heloderma suspectum)-(1-39)-peptidylpenta-L-lysyl-L-lysinamide (CAS number 320367-13-3).

The aim of the present invention can be seen in the improvement of anti-diabetic treatment in children and adolescents suffering from diabetes mellitus, in particular from type 2 diabetes mellitus.

In Examples 1 and 2 of the present invention, after a standardized liquid breakfast in 12 pediatric patients with Type 2 Diabetes mellitus (T2DM) with a mean HbA_{1c} of 8.65% and mean body weight of 84.7 kg, a non-significant decrease in plasma glucose (corrected plasma glucose AUC_{0:30h-4:30h} and plasma glucose AUC_{0:30h-4:30h}) was observed with single doses of lixisenatide 5 and 10 μg compared to placebo. In contrast, single doses of lixisenatide 5 and 10 μg significantly reduced plasma glucose (corrected plasma glucose AUC_{0:30h-4:30h} and plasma glucose AUC_{0:30h-4:30h}) compared to placebo in 12 adult patients with T2DM. Lixisenatide exposure was similar for both dose groups in the evaluable pediatric patients, whereas in adult patients, the lixisenatide exposure dose-proportionally increased. In pediatric patients, the exposure was similar to that in adults for lixisenatide 5 μg, but lower for lixisenatide 10 μg. Single doses of lixisenatide 5 and 10 μg were safe and well tolerated in both, pediatric and adult patients in this study of short duration.

In conclusion, Examples 1 and 2 demonstrated comparable pharmacokinetic (PK) and pharmacodynamic (PD) profiles in pediatric and adults patients at a dose of 5 μg, as well as no unexpected safety results.

Examples 1 and 2 of the present invention confirm that adult patients have a different pathophysiology compared with children and adolescents. The response to a standardized liquid breakfast differed in pediatric type 2 diabetes mellitus patients from that of adult patients. In the pediatric control population (placebo group), the peak insulin concentration was observed about one hour after the test meal (FIG. 8), followed by a rapid decline. In the adult control population (placebo group), the postprandial insulin peak was broader. The peak insulin concentration was observed about 2 hours after the test meal (FIG. 7). A similar difference was observed in the postprandial C-peptide concentration (FIGS. 9 and 10).

In line with these findings, differences were identified in the effects of lixisenatide in the pediatric patients compared with the effects obtained in the adult patient population. The above-described differences in time course of postprandial insulin and C-peptide secretion found in the placebo groups were also observed under lixisenatide.

The exposure of lixisenatide in pediatric patients lixisenatide was smaller than in adults at doses of 10 μg (FIGS. 11 and 12). The reduction of postprandial plasma glucose (PPG) by treatment with lixisenatide was smaller than in adult patients, and, due to the small number of patients, not significant (FIGS. 1 to 4).

Surprisingly, at a dose of 5 μg, lixisenatide exhibited a larger reduction in plasma glucacon level than in adult patients (FIGS. 5 and 6).

In summary, the results of Example 2 indicate differences in the pathophysiology of pediatric type 2 diabetes mellitus patients and adult patients. The fact that lixisenatide can reduce postprandial plasma glucose, postprandial glucagon and insulin secretion in pediatric patients indicates that lixisenatide is effective in the treatment of this patient group.

Example 3 of the present invention describes a randomized, double-blind, placebo-controlled, dose escalation, study on safety, pharmacokinetics and pharmacodynamics of lixisenatide in pediatric patients with type 2 diabetes not adequately controlled with metformin and/or basal insulin.

A first aspect of the present invention is lixisenatide or/and a pharmaceutically acceptable salt thereof, for use in pediatrics.

Another aspect of the present invention is a pharmaceutical combination for use in pediatrics, said combination comprising
(a) lixisenatide or/and a pharmaceutically acceptable salt thereof, and
(b) metformin or/and a pharmaceutically acceptable salt thereof.

Yet another aspect of the present invention is a pharmaceutical combination for use in paediatrics, said combination comprising
(a) lixisenatide and/or a pharmaceutically acceptable salt thereof,
(b) metformin or/and a pharmaceutically acceptable salt thereof, and
(c) basal insulin or/and a pharmaceutically acceptable salt thereof.

Yet another aspect of the present invention is a pharmaceutical combination for use in paediatrics, said combination comprising
(a) lixisenatide or/and a pharmaceutically acceptable salt thereof, and
(b) a basal insulin or/and a pharmaceutically acceptable salt thereof.

As used herein, “to be treated according to the present invention”, “treatment according to the present invention”, or “pediatric treatment according to the present invention” relates to the treatment of pediatric patients, as defined herein, by (i) lixisenatide or/and a pharmaceutically acceptable salt thereof, or (ii) the pharmaceutical combination as described herein.

The patient in need of the pediatric treatment according to the present invention by may have an age of at least 10 years.

The patient in need of the pediatric treatment according to the present invention as described herein may have an age of less than 18 years.

It is preferred that the patient in need of the pediatric treatment according to the present invention as described herein may have (a) an age of at least 10 years, and (b) and age of less than 18 years.

The patient in need of the pediatric treatment according to the present invention as described herein may suffer from type 2 diabetes mellitus.

The pediatric patient to be treated according to the present invention may be a subject suffering from type 2 diabetes mellitus, wherein type 2 diabetes mellitus is not adequately controlled by treatment with metformin monotherapy, for instance with a dose of at least 1.0 g/day metformin or at least 1.5 g/day metformin for 3 months, or/and a dose of at the maximum 2.0 g/day metformin for 3 months.

The pediatric patient to be treated according to the present invention may be a subject suffering from type 2 diabetes mellitus, wherein type 2 diabetes mellitus is not adequately controlled by treatment with a basal insulin or/and metformin, for instance with a dose of at least 1.0 g/day metformin or at least 1.5 g/day metformin for 3 months, or/and a dose of at the maximum 2.0 g/day metformin for 3 months.

The pediatric patient to be treated according to the present invention may be a subject suffering from type 2 diabetes mellitus, where type 2 diabetes mellitus snot adequately controlled by treatment with a basal insulin monotherapy.

In the present invention, “not adequately controlled” by the treatment with metformin monotherapy (treatment with metformin alone) means that metformin monotherapy is not sufficient to remove the symptoms of diabetes mellitus. In particular, “not adequately controlled” by the treatment with metformin monotherapy means that the patient does not reach normoglycemic values in terms of, for example, postprandial plasma glucose concentration, glucose excursion or/and fasting plasma glucose concentration.

In the present invention, “not adequately controlled” by the treatment with metformin or/and a basal insulin means that this therapy alone is not sufficient to remove the symptoms of diabetes mellitus. In particular, “not adequately controlled” by the treatment with metformin or/and a basal insulin means that the patient does not reach normoglycemic values in terms of, for example, postprandial plasma glucose concentration, glucose excursion or/and fasting plasma glucose concentration.

In the present invention, “not adequately controlled” by the treatment with a basal insulin monotherapy (treatment with a basal insulin alone) means that this therapy alone is not sufficient to remove the symptoms of diabetes mellitus. In particular, “not adequately controlled” by the treatment with a basal insulin monotherapy means that the patient does not reach normoglycemic values in terms of, for example, postprandial plasma glucose concentration, glucose excursion or/and fasting plasma glucose concentration.

The term “not adequately controlled” by the treatment with metformin monotherapy in particular relates to the period before onset of treatment according to the present invention. It can be diagnosed before onset of the treatment according to the present invention if monotherapy with metformin adequately controls the type 2 diabetes mellitus or not. For example, such diagnosis may be performed within 1 months, within 2 months or within 3 months before onset of the therapy of the present invention.

The term “not adequately controlled” by the treatment with metformin or/and a basal insulin in particular relates to the period before onset of treatment according to the present invention. It can be diagnosed before onset of the treatment according to the present invention if the therapy with metformin or/and a basal insulin adequately controls the type 2 diabetes mellitus or not. For example, such diagnosis may be performed within 1 months, within 2 months or within 3 months before onset of the therapy of the present invention.

The term “not adequately controlled” by the treatment with a basal insulin monotherapy in particular relates to the period before onset of treatment according to the present invention. It can be diagnosed before onset of the treatment according to the present invention if the therapy with a basal insulin monotherapy adequately controls the type 2 diabetes mellitus or not. For example, such diagnosis may be performed within 1 months, within 2 months or within 3 months before onset of the therapy of the present invention.

By the treatment according to the present invention, adequate control of type 2 diabetes mellitus may be achieved in pediatric patients not adequately controlled with metformin monotherapy.

By the treatment according to the present invention, adequate control of type 2 diabetes mellitus may be achieved in pediatric patients not adequately controlled with metformin or/and a basal insulin.

By the treatment according to the present invention, adequate control of type 2 diabetes mellitus may be achieved in pediatric patients not adequately controlled with a basal insulin monotherapy.

The pediatric patient suffering from type 2 diabetes mellitus to be treated according to the present invention may be obese. A patient can be considered as obese if the body mass index is at least 30 kg/m². In the present invention, an obese pediatric patient may have a body mass index of at least 30 kg/m² or at least 31 kg/m². It is preferred that that the pediatric patient has a body mass index of at least 31 kg/m².

The pediatric patient suffering from type 2 diabetes mellitus to be treated according to the present invention preferably does not receive an antidiabetic treatment by insulin or/and related compounds.

The pediatric patient suffering from type 2 diabetes mellitus to be treated according to the present invention may suffer from type 2 diabetes mellitus for at least three months. In particular, in the pediatric patient to be treated, type 2 diabetes mellitus has been diagnosed for at least three months before onset of therapy of the present invention.

In the present invention, a pediatric patient may have a HbA1c value in the range of 7% to 10%, or 7% to 9.9%. In particular the pediatric patient to be treated may have a HbA_{1c} value of at least about 7%, at least about 7.5%, at least about 8%, at least about 8.5%, at least about 8.65%, or at least about 9%.

In particular, in a pediatric patient receiving metformin monotherapy (in particular before onset of therapy according to the present invention), a HbA1c value in the range of 7% to 10% or 7% to 9.9%, or a HbA_{1c} value of at least about 7%, at least about 7.5%, at least about 8%, at least about 8.5%, at least about 8.65%, or at least about 9% indicates that the type 2 diabetes mellitus is not adequately controlled by metformin monotherapy.

In particular, in a pediatric patient receiving metformin or/and a basal insulin (in particular before onset of therapy according to the present invention), a HbA1c value in the range of 7% to 10% or 7% to 9.9%, or a HbA_{1c} value of at least about 7%, at least about 7.5%, at least about 8%, at least about 8.5%, at least about 8.65%, or at least about 9% indicates that the type 2 diabetes mellitus is not adequately controlled by metformin or/and a basal insulin.

In particular, in a pediatric patient receiving a basal insulin monotherapy (in particular before onset of therapy according to the present invention), a HbA1c value in the range of 7% to 10% or 7% to 9.9%, or a HbA_{1c} value of at least about 7%, at least about 7.5%, at least about 8%, at least about 8.5%, at least about 8.65%, or at least about 9% indicates that the type 2 diabetes mellitus is not adequately controlled by a basal insulin monotherapy.

In the present invention, normoglycemic values are blood glucose concentrations of in particular 60-140 mg/dl (corresponding to 3.3 to 7.8 mmol/L). This range refers in particular to blood glucose concentrations under fasting conditions and postprandial conditions.

Criteria for a type 2 diabetes mellitus diagnosis include:
the fasting plasma glucose concentration (FPG) is ≥7.0 mmol/L (126 mg/dl), or
the post challenge plasma glucose concentration is >11.1 mmol/L (200 mg/dl), performed as described by the World Health Organization (Definition, Diagnosis and Classification of Diabetes Mellitus and its Complications. Part 1: Diagnosis and Classification of Diabetes Mellitus. WHO/NCD/NCS/99.2. Geneva; 1999), using a glucose load containing the equivalent of 75 g anhydrous glucose dissolved in water, or
symptoms of diabetes and a casual plasma glucose ≥200 mg/dl (11.1 mmol/L).

These criteria are described in the Global IDF/ISPAD Guideline for Diabetes in Childhood and Adolescence (International Diabetes Federation, ISBN 2-930229-72-1).

The diagnosis of Type 2 Diabetes should not be based on a single plasma glucose concentration. Diagnosis may require continued observation with fasting and/or postprandial blood glucose levels and/or an oral glucose tolerance test.

According to Craig (Pediatric Diabetes 2014: 15(Suppl. 20): 4-17), fasting plasma glucose (FPG) and post challenge (postload) glucose can be classified as follows:
FPG<5.6 mmol/L (100 mg/dL)=normal fasting glucose concentration.
FPG 5.6 to 6.9 mmol/L (100-125 mg/dL)=impaired fasting glucose concentration.
FPG≥7.0 mmol/L (126 mg/dL)=provisional diagnosis of diabetes (the diagnosis must be confirmed, as described above)

The corresponding categories when the Oral Glucose Tolerance Test (OGTT) is used are as follows:
Two hour postload glucose <7.8 mmol/L (140 mg/dL)=normal glucose tolerance.
Two hour postload glucose 7.8 to <11.1 mmol/L (140-200 mg/dL)=impaired glucose tolerance.
Two hour postload glucose ≥11.1 mmol/L (200 mg/dL)=provisional diagnosis of diabetes (the diagnosis must be confirmed, as described above).

Impaired glucose tolerance (IGT) and impaired fasting glucose concentration (IFG) are intermediate stages in the natural history of disordered carbohydrate metabolism between normal glucose homeostasis and diabetes.

In the present invention, normoglycemic glucose concentrations can include impaired glucose concentrations, as described herein.

In the present invention, normoglycemic values of fasting plasma glucose are blood glucose concentrations of in particular <5.6 mmol/L or <7.0 mmol/L.

In the present invention, normoglycemic values of postprandial plasma glucose, as defined herein, are blood glucose concentrations of in particular <7.8 mmol/L or <11.1 mmol/L.

The pediatric patient to be treated according to the present invention may have a 2 hours postprandial plasma glucose concentration of at least 11.1 mmol/L, at least 12 mmol/L, or at least 13 mmol/L. These plasma glucose concentrations exceed normoglycemic concentrations.

In particular, in a pediatric patient receiving metformin monotherapy (in particular before onset of therapy according to the present invention), a 2 hours postprandial plasma concentration of at least 11.1 mmol/L, at least 12 mmol/L or at least 13 mmol/L indicates that the type 2 diabetes mellitus is not adequately controlled by metformin monotherapy.

In particular, in a pediatric patient receiving metformin or/and a basal insulin (in particular before onset of therapy according to the present invention), a 2 hours postprandial plasma concentration of at least 11.1 mmol/L, at least 12 mmol/L or at least 13 mmol/L indicates that the type 2 diabetes mellitus is not adequately controlled by metformin or/and a basal insulin.

In particular, in a pediatric patient receiving a basal insulin monotherapy (in particular before onset of therapy according to the present invention), a 2 hours postprandial plasma concentration of at least 11.1 mmol/L, at least 12 mmol/L or at least 13 mmol/L indicates that the type 2 diabetes mellitus is not adequately controlled by a basal insulin monotherapy.

“Postprandial” is a term that is well known to a person skilled in the art of diabetology. The term “postprandial” describes in particular the phase after an ingestion of a meal or/and exposure to glucose under experimental conditions. In a healthy person this phase is characterised by an increase and subsequent decrease in blood glucose concentration. The postprandial phase typically ends up to 2 h after a meal or/and exposure to glucose.

Determination of postprandial plasma glucose is well-known (see, e.g. Capro et al., Diabetes, 1977, 26(12):1178-1183). A typical standardized breakfast suitable for exposure to glucose under experimental conditions in a meal test is described in the Appendix of Example 2.

The pediatric patient to be treated according to the invention may have a glucose excursion of at least 3 mmol/L, at least 3.5 mmol/L or at least 3.65 mmol/L. In the present invention, the glucose excursion is in particular the difference of the 2 hours postprandial plasma glucose concentration and the plasma glucose concentration prior to a meal test, e.g. the plasma glucose concentration 30 minutes prior to a meal test.

In particular, in a pediatric patient receiving metformin monotherapy (in particular before onset of therapy according to the present invention), a glucose excursion of at least 3 mmol/L, at least 3.5 mmol/L or at least 3.65 mmol/L indicates that the type 2 diabetes mellitus is not adequately controlled by metformin monotherapy.

In particular, in a pediatric patient receiving metformin or/and a basal insulin (in particular before onset of therapy according to the present invention), a glucose excursion of at least 3 mmol/L, at least 3.5 mmol/L or at least 3.65 mmol/L indicates that the type 2 diabetes mellitus is not adequately controlled by metformin or/and a basal insulin.

In particular, in a pediatric patient receiving a basal insulin monotherapy (in particular before onset of therapy according to the present invention), a glucose excursion of at least 3 mmol/L, at least 3.5 mmol/L or at least 3.65 mmol/L indicates that the type 2 diabetes mellitus is not adequately controlled by a basal insulin monotherapy.

The pediatric patient to be treated according to the invention may have a fasting plasma glucose concentration of at least 8 mmol/L, or at least 8.5 mmol/L. These plasma glucose concentrations exceed normoglycemic concentrations.

In particular, in a pediatric patient receiving metformin monotherapy (in particular before onset of therapy according to the present invention), a fasting plasma glucose concentration of at least 8 mmol/L, or at least 8.5 mmol/L indicates that the type 2 diabetes mellitus is not adequately controlled by metformin monotherapy.

In particular, in a pediatric patient receiving metformin or/and a basal insulin (in particular before onset of therapy according to the present invention), a fasting plasma glucose concentration of at least 8 mmol/L, or at least 8.5 mmol/L indicates that the type 2 diabetes mellitus is not adequately controlled by metformin or/and a basal insulin.

In particular, in a pediatric patient receiving basal insulin monotherapy (in particular before onset of therapy according to the present invention), a fasting plasma glucose concentration of at least 8 mmol/L, or at least 8.5 mmol/L indicates that the type 2 diabetes mellitus is not adequately controlled by basal insulin monotherapy.

The pediatric patient to be treated according to the invention may have a C-peptide plasma concentration of at least 1.2 nmol/L in fasting conditions.

The pediatric patient to be treated according to the invention may have a plasma glucagon level of at least 140 ng/L in fasting conditions.

In another aspect of the present invention, (i) lixisenatide or/and a pharmaceutically acceptable salt thereof, or (ii) the combination as described herein can be used for improving (i.e. reducing) the 2 hours postprandial plasma glucose in a pediatric patient suffering from type 2 diabetes mellitus. Reduction means in particular that the 2 hours postprandial plasma glucose concentration reaches normoglycemic values or at least approaches these values.

In another aspect of the present invention, (i) lixisenatide or/and a pharmaceutically acceptable salt thereof, or (ii) the combination as described herein can be used for improving (i.e. reducing) the glucose excursion in a pediatric patient suffering from type 2 diabetes mellitus. Reduction means in particular that the glucose excursion reaches normoglycemic values or at least approaches these values.

In another aspect of the present invention, (i) lixisenatide or/and a pharmaceutically acceptable salt thereof, or (ii) the combination as described herein can be used for improving (i.e. reducing) the plasma glucagon concentration in a pediatric patient suffering from type 2 diabetes mellitus.

Lixisenatide or/and a pharmaceutically acceptable salt thereof, or the combination of the present invention can be used in the treatment of one or more of the medical indications described herein, for example in treatment of type 2 diabetes mellitus patients, as described herein, or for conditions associated with type 2 diabetes mellitus, such as for the improvement of glucose excursion, for improvement of the postprandial plasma glucose concentration, or/and for improvement of plasma glucagon concentration.

The plasma glucagon concentration, as used herein, is in particular the postprandial plasma glucagon concentration.

In the present invention, metformin includes pharmaceutically acceptable salts thereof. The person skilled in the art knows suitable pharmaceutically acceptable salts of metformin.

In the present invention, metformin can be administered according to commonly known administration protocols of metformin in accordance with the terms of marketing authorization. Metformin can be administered to patients from 10 years. For example, metformin can be administrated once daily, twice daily or three times a day. In particular, the metformin dose applied before the onset of the therapy as disclosed herein is continued in combination with lixisenatide or/and a pharmaceutically acceptable salt thereof, as disclosed herein.

In the present invention, metformin may be administered orally. The skilled person knows formulations of metformin suitable for treatment of type 2 diabetes mellitus by oral administration. Metformin may be administered to a pediatric patient in need thereof, in an amount sufficient to induce a therapeutic effect. Metformin may be administered in a dose of at least 1.0 g/day or at least 1.5 g/day. Metformin may be administered in a dose of at the maximum of 2.0 g/day. The daily metformin dose can be divided into 2 or three separate doses. For oral administration, metformin may be formulated in a solid dosage form, such as a tablet or pill. Metformin may be formulated with suitable pharmaceutically acceptable carriers, adjuvants, or/and auxiliary substances.

In the present invention, lixisenatide or/and a pharmaceutically acceptable salt may be administered in an add-on therapy to administration of metformin.

In the present invention, the terms “add-on”, “add-on treatment” and “add-on therapy” can relate to treatment according to the present invention with metformin and lixisenatide.

In the present invention, the terms “add-on”, “add-on treatment” and “add-on therapy” can also relate to treatment according to the present invention with a basal insulin or/and metformin, and lixisenatide.

In the present invention, the terms “add-on”, “add-on treatment” and “add-on therapy” can also relate to treatment according to the present invention with a basal insulin and lixisenatide.

Metformin, lixisenatide or/and the basal insulin each may be administered in a once-a-day-dosage. Metformin, the basal insulin and lixisenatide may be administered by different administration routes. Metformin may be administered orally, and lixisenatide and the basal insulin may be administered parenterally.

In particular, “add-on”, “add-on treatment” and “add-on therapy” mean that the dose of metformin administered before the onset of the treatment with lixisenatide or/and a pharmaceutically acceptable salt thereof, as disclosed herein, is continued in combination with lixisenatide or/and a pharmaceutically acceptable salt thereof.

In particular, “add-on”, “add-on treatment” and “add-on therapy” mean that the dose of the basal insulin administered before the onset of the treatment with lixisenatide or/and a pharmaceutically acceptable salt thereof, as disclosed herein, is continued in combination with lixisenatide or/and the pharmaceutically acceptable salt thereof. Alternatively, the dose of the basal insulin may be reduced in combination with lixisenatide or/and the pharmaceutically acceptable salt thereof.

In the present invention, lixisenatide includes pharmaceutically acceptable salts thereof. The person skilled in the art knows suitable pharmaceutically acceptable salts of lixisenatide. A preferred pharmaceutically acceptable salt of lixisenatide employed in the present invention is the acetate salt of lixisenatide.

In the present invention, lixisenatide or/and the pharmaceutically acceptable salt thereof may be administered to a pediatric patient in need thereof, in an amount sufficient to induce a therapeutic effect.

In the present invention, lixisenatide or/and the pharmaceutically acceptable salt thereof may be formulated with suitable pharmaceutically acceptable carriers, adjuvants, or/and auxiliary substances.

Lixisenatide or/and a pharmaceutically acceptable salt thereof may be administered parenterally, e.g. by injection (such as by intramuscular or by subcutaneous injection). Suitable injection devices, for instance the so-called “pens” comprising a cartridge comprising the active ingredient, and an injection needle, are known. Lixisenatide or/and a pharmaceutically acceptable salt thereof may be administered in a suitable amount, for instance in an amount in the range of 5 μg to 10 μg per dose.

In the present invention, lixisenatide or/and a pharmaceutically acceptable salt thereof may be administered in a daily dose in the range of 5 to 10 μg. Lixisenatide or/and a pharmaceutically acceptable salt thereof may be administered by one injection per day. Lixisenatide or/and a pharmaceutically acceptable salt thereof may be administered about 30 min before breakfast.

In the present invention, lixisenatide or/and a pharmaceutically acceptable salt thereof may be provided in a liquid composition, which preferably is an aqueous formulation. It is preferred that the liquid composition is suitable for parenteral administration, in particular for injection. The skilled person knows such liquid compositions of lixisenatide. A liquid composition of the present invention may have an acidic or a physiologic pH. An acidic pH preferably is in the range of pH 1-6.8, pH 3.5-6.8, or pH 3.5-5. A physiologic pH preferably is in the range of pH 2.5-8.5, pH 4.0-8.5, or pH 6.0-8.5. The pH may be adjusted by a pharmaceutically acceptable diluted acid (typically HCl) or pharmaceutically acceptable diluted base (typically NaOH).

The liquid composition comprising lixisenatide or/and a pharmaceutically acceptable salt thereof may comprise a suitable preservative. A suitable preservative may be selected from phenol, m-cresol, benzyl alcohol and p-hydroxybenzoic acid ester. A preferred preservative is m-cresol.

The liquid composition comprising lixisenatide or/and a pharmaceutically acceptable salt thereof may comprise a tonicity agent. A suitable tonicity agent may be selected from glycerol, lactose, sorbitol, mannitol, glucose, NaCl, calcium or magnesium containing compounds such as CaCl₂). The concentration of glycerol, lactose, sorbitol, mannitol and glucose may be in the range of 100-250 mM. The concentration of NaCl may be up to 150 mM. A preferred tonicity agent is glycerol.

The liquid composition comprising lixisenatide or/and a pharmaceutically acceptable salt thereof may comprise methionine from 0.5 μg/mL to 20 μg/mL, preferably from 1 μg/ml to 5 μg/ml. Preferably, the liquid composition comprises L-methionine.

In the present invention, the basal insulin includes pharmaceutically acceptable salts thereof. The person skilled in the art knows suitable pharmaceutically acceptable salts.

In the present invention, the basal insulin or/and the pharmaceutically acceptable salt thereof may be administered to a pediatric patient in need thereof, in an amount sufficient to induce a therapeutic effect.

In the present invention, the basal insulin or/and the pharmaceutically acceptable salt thereof may be formulated with suitable pharmaceutically acceptable carriers, adjuvants, or/and auxiliary substances.

The basal insulin or/and a pharmaceutically acceptable salt thereof may be administered parenterally, e.g. by injection (such as by intramuscular or by subcutaneous injection). Suitable injection devices, for instance the so-called “pens” comprising a cartridge comprising the active ingredient, and an injection needle, are known.

A further aspect of the present invention is a method of pediatric treatment, said method comprising administering to a patient in need of a pediatric treatment, lixisenatide or/and a pharmaceutically acceptable salt thereof.

In this method of treatment, the pediatric patient is a patient as described herein. In particular, the pediatric patient suffers from type 2 diabetes mellitus, as described herein. Lixisenatide is prepared as described herein, in particular as a liquid formulation suitable for parenteral administration.

Another aspect of the present invention is a method of pediatric treatment, said method comprising administering to a patient in need of a pediatric treatment, a pharmaceutical combination, said combination comprising
(a) lixisenatide or/and a pharmaceutically acceptable salt thereof, and
(b) metformin or/and a pharmaceutically acceptable salt thereof.

Another aspect of the present invention is a method of pediatric treatment, said method comprising administering to a patient in need of a pediatric treatment, a pharmaceutical combination, said combination comprising
(a) lixisenatide or/and a pharmaceutically acceptable salt thereof, and
(b) a basal insulin or/and a pharmaceutically acceptable salt thereof.

Another aspect of the present invention is a method of pediatric treatment, said method comprising administering to a patient in need of a pediatric treatment, a pharmaceutical combination, said combination comprising
(a) lixisenatide or/and a pharmaceutically acceptable salt thereof
(b) metformin or/and a pharmaceutically acceptable salt thereof, and
(c) a basal insulin or/and a pharmaceutically acceptable salt thereof.

In these methods of treatment, the pediatric patient is a patient as described herein. In particular, the pediatric patient suffers from type 2 diabetes mellitus, as described herein. Lixisenatide is prepared as described herein, in particular as a liquid formulation suitable for parenteral administration. Metformin is prepared as described herein, in particular for oral administration. The basal insulin is prepared as described herein, in particular as a liquid formulation suitable for parenteral administration.

Yet another aspect of the present invention is a method for the improvement of glucose excursion, for the improvement of the postprandial plasma glucose concentration, or/and for the improvement of plasma glucagon concentration, said method comprising administering to a pediatric patient, as described herein, (i) lixisenatide or/and a pharmaceutically acceptable salt thereof, or (ii) the combination as described herein.

Yet another aspect of the present invention is the use of lixisenatide or/and a pharmaceutically acceptable salt thereof, for the manufacture of a medicament for use in pediatrics (for pediatric treatment). The pediatric patient is a patient as described herein. In particular, the medicament is for the treatment of type 2 diabetes mellitus, as described herein. Lixisenatide is prepared as described herein, in particular as a liquid formulation suitable for parenteral administration.

Yet another aspect of the present invention is the use of a pharmaceutical combination, said combination comprising
(a) lixisenatide or/and a pharmaceutically acceptable salt thereof, and
(b) metformin or/and a pharmaceutically acceptable salt thereof,
for the manufacture of a medicament for use in pediatrics (for pediatric treatment).

Yet another aspect of the present invention is the use of a pharmaceutical combination, said combination comprising
(a) lixisenatide or/and a pharmaceutically acceptable salt thereof, and
(b) a basal insulin or/and a pharmaceutically acceptable salt thereof,
for the manufacture of a medicament for use in pediatrics (for pediatric treatment).

Yet another aspect of the present invention is the use of a pharmaceutical combination, said combination comprising
(a) lixisenatide or/and a pharmaceutically acceptable salt thereof,
(b) metformin or/and a pharmaceutically acceptable salt thereof, and
(c) a basal insulin or/and a pharmaceutically acceptable salt thereof,
for the manufacture of a medicament for use in pediatrics (for pediatric treatment).

In these uses, the pediatric patient is a patient as described herein. In particular, the medicament is for the treatment of type 2 diabetes mellitus, as described herein. Lixisenatide is prepared as described herein, in particular as a liquid formulation suitable for parenteral administration. Metformin is prepared as described herein, in particular for oral administration. The basal insulin is prepared as described herein, in particular as a liquid formulation suitable for parenteral administration.

Yet another aspect of the present invention is the use of (i) lixisenatide or/and a pharmaceutically acceptable salt thereof, or (ii) the combination as described herein, for the manufacture of a medicament for the improvement of glucose excursion, for the improvement of the postprandial plasma glucose concentration, or/and for the improvement of plasma glucagon concentration, wherein the patient to be treated is a pediatric patient, as described herein.

The invention is further illustrated by the following examples and figures.

### BRIEF DESCRIPTION OF THE DRAWINGS

FIG. 1 Mean±SEM plasma glucose per treatment group in adult patients−evaluable PD population

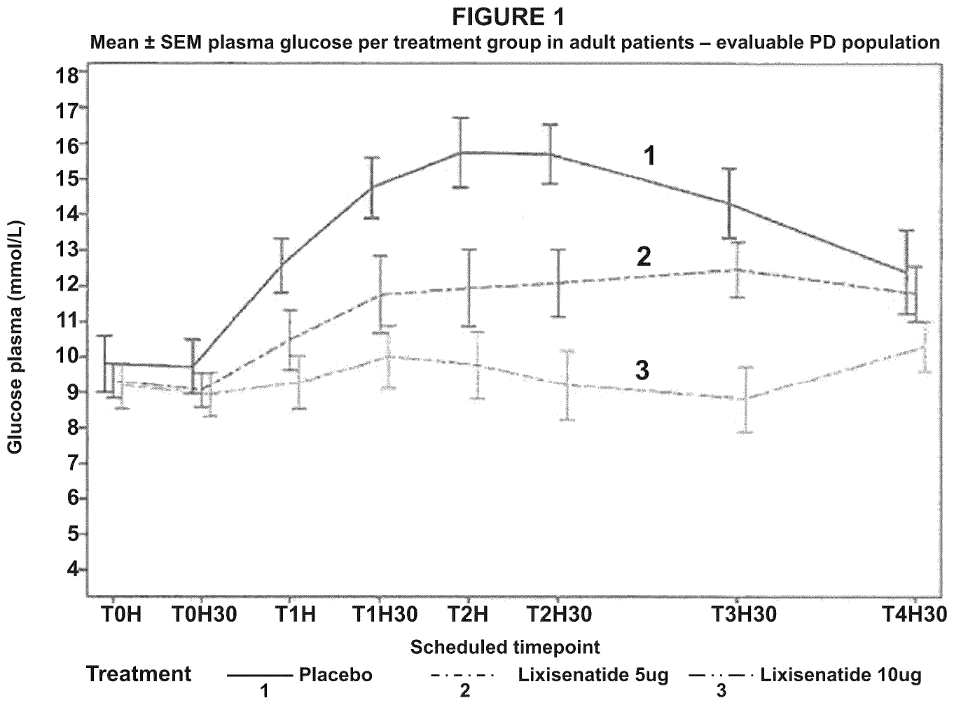

FIG. 2 Mean±SEM plasma glucose per treatment group in pediatric patients−evaluable PD population

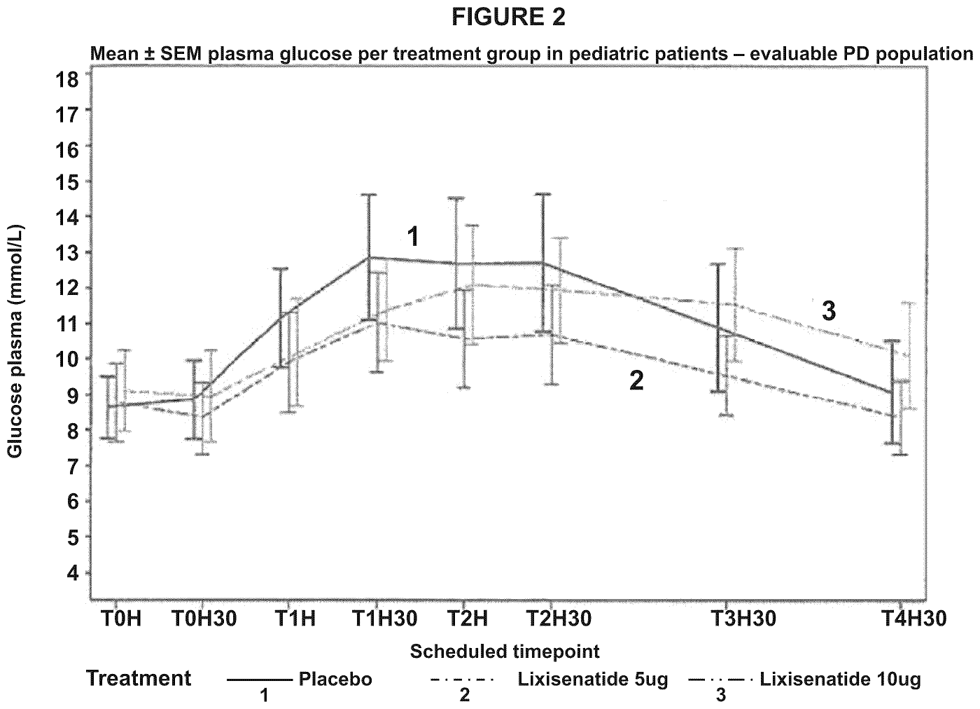

FIG. 3 Median plasma glucose (mmol/L) per treatment group in adult patients−evaluable PD population

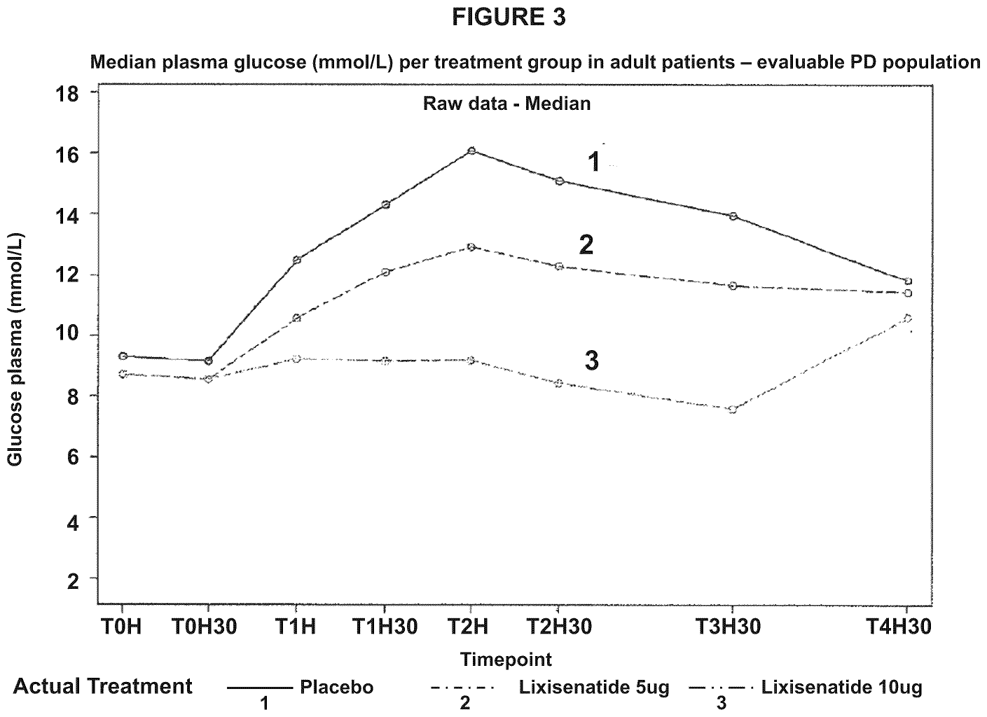

FIG. 4 Median plasma glucose (mmol/L) per treatment group in pediatric patients−evaluable PD population

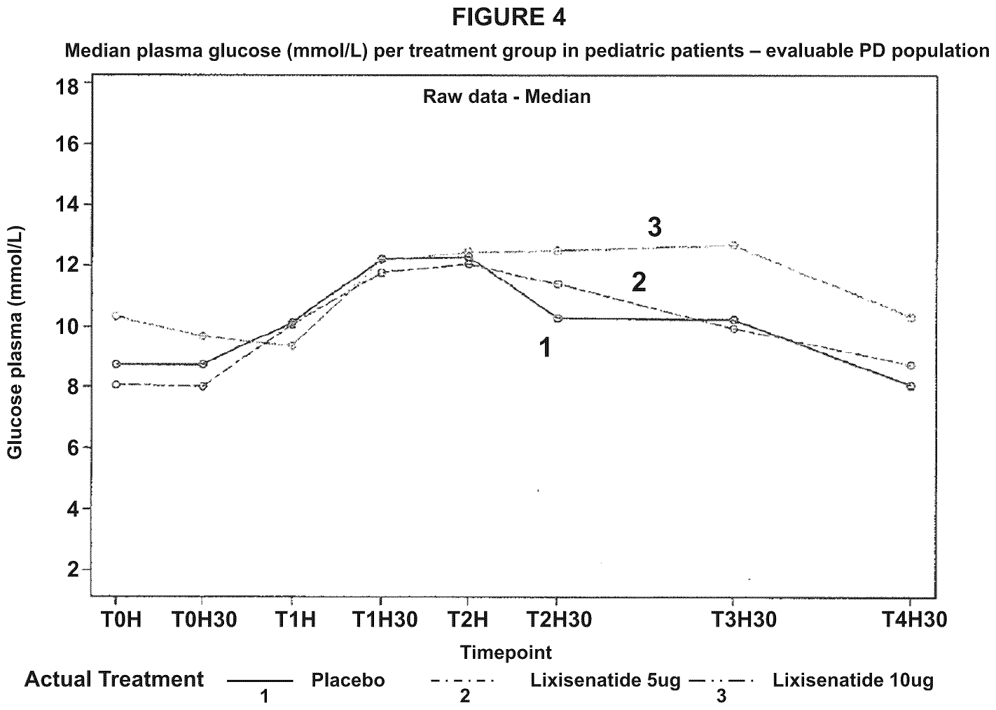

FIG. 5 Median glucagon (ng/L) per treatment group in adult patients−evaluable PD population

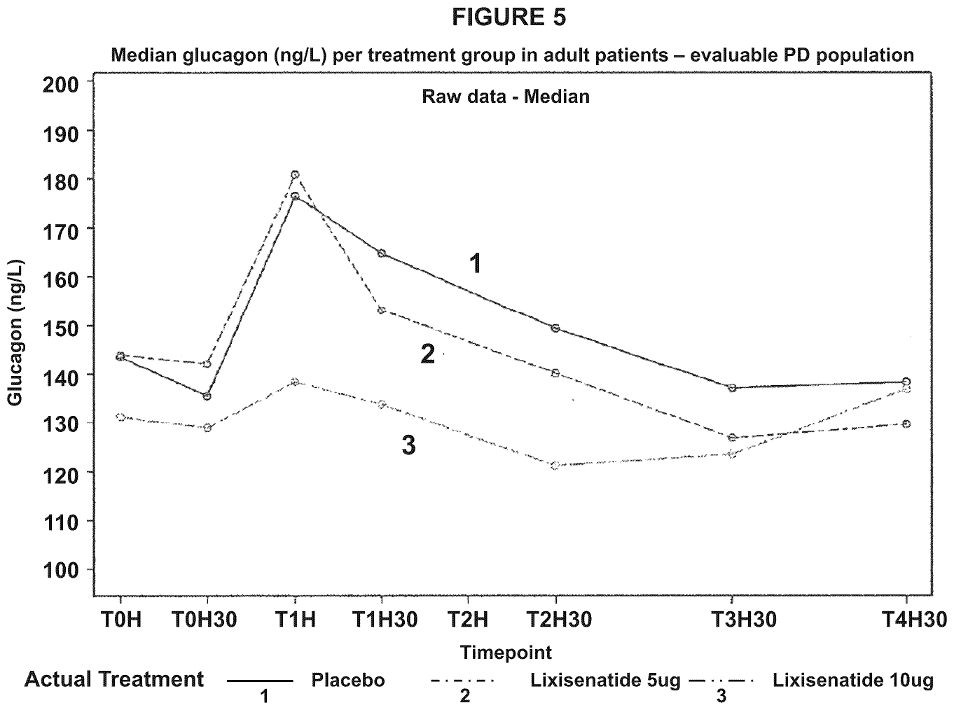

FIG. 6 Median glucagon (ng/L) per treatment group in pediatric patients−evaluable PD population

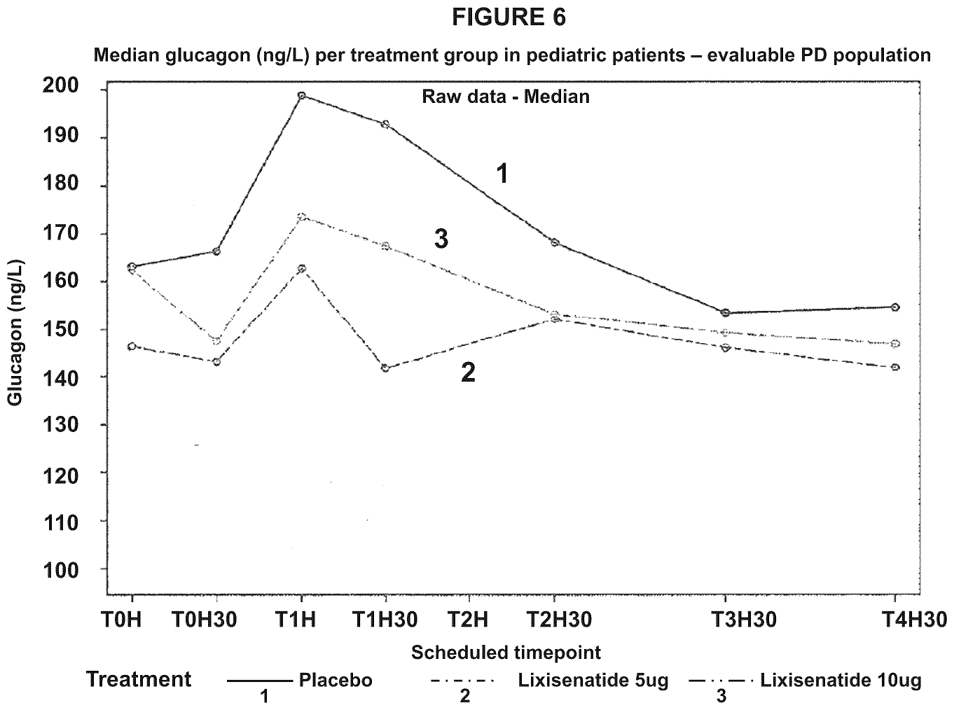

FIG. 7 Median plasma insulin (pmol/L) per treatment group in adult patients−evaluable PD population

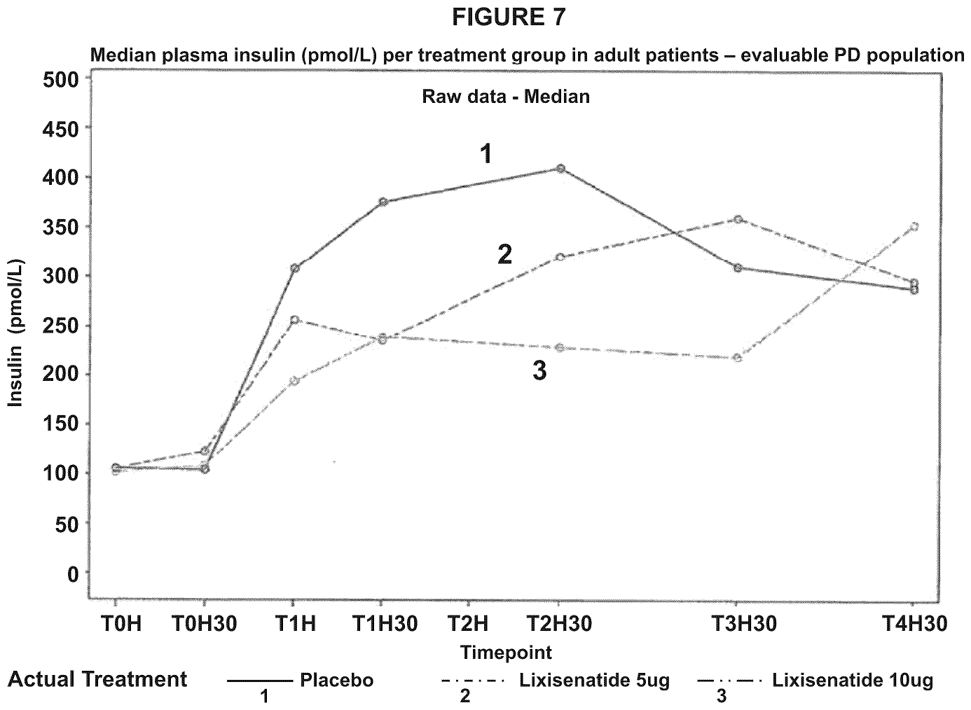

FIG. 8 Median plasma insulin (pmol/L) per treatment group in pediatric patients−evaluable PD population

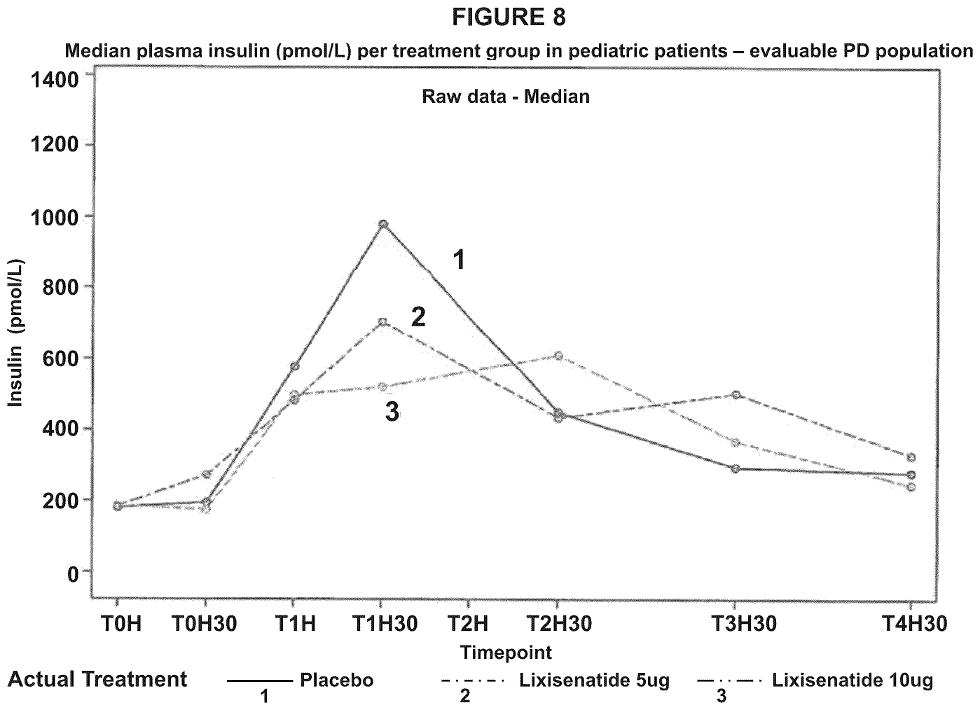

FIG. 9 Median C-peptide (nmol/L) per treatment group in adult patients−evaluable PD population

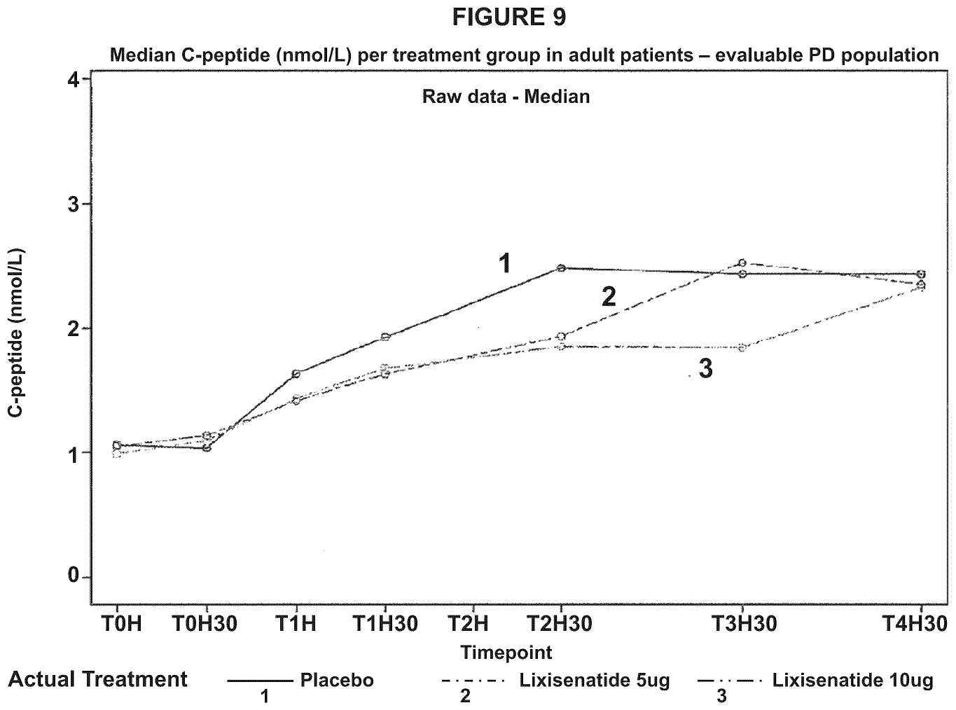

FIG. 10 Median C-peptide (nmol/L) per treatment group in pediatric patients−evaluable PD population

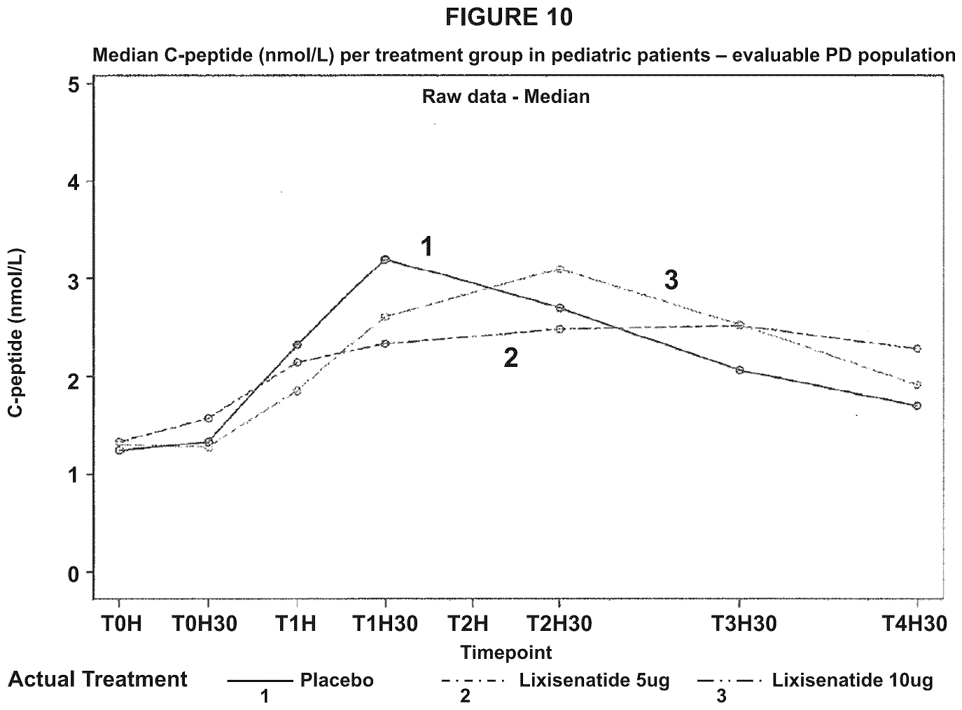

FIG. 11 Mean (+SD) lixisenatide plasma concentrations by treatment (full PK population, linear scale)

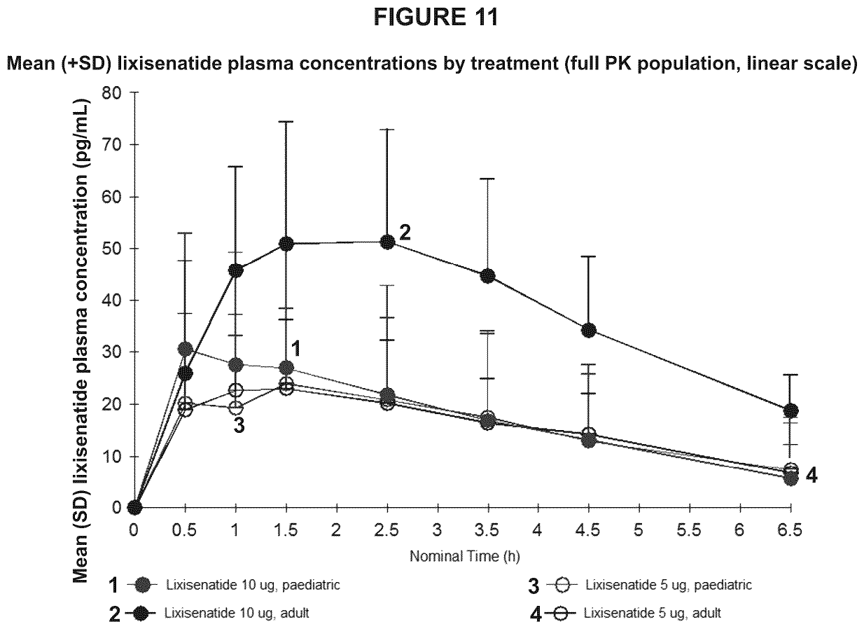

FIG. 12 Mean (+SD) lixisenatide plasma concentrations by treatment (evaluable PK population, linear scale)

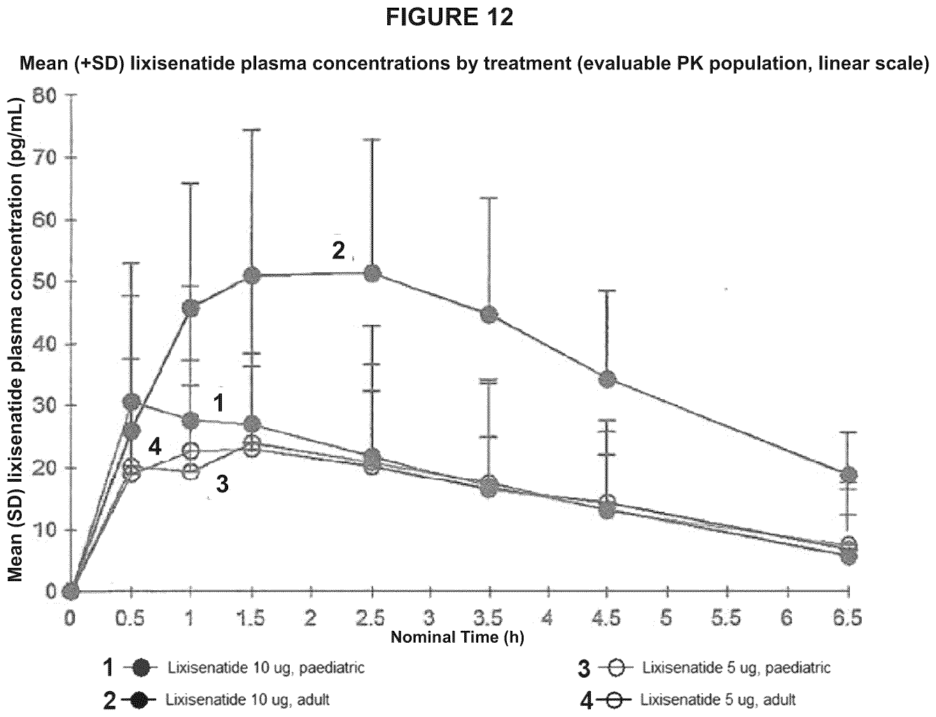

FIG. 13 Graphical study design

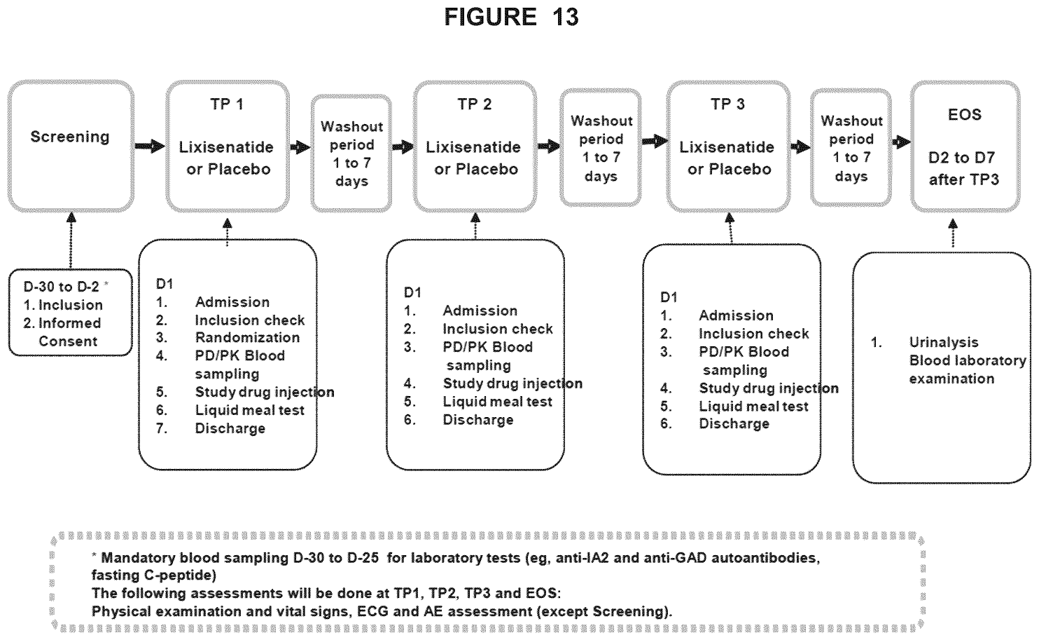

FIG. 14 shows a representation of 3 cartridges for injections

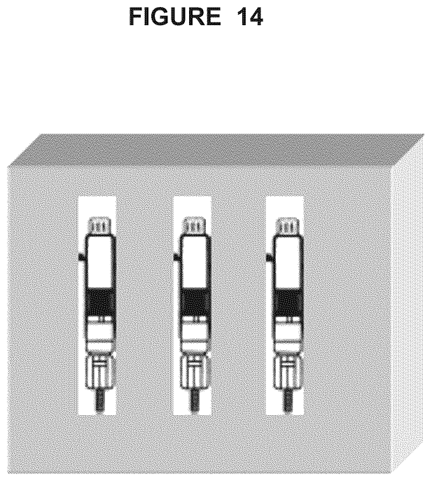

FIG. 15 shows Body Mass Index (BMI) for age percentiles for boys 2 to 20 years

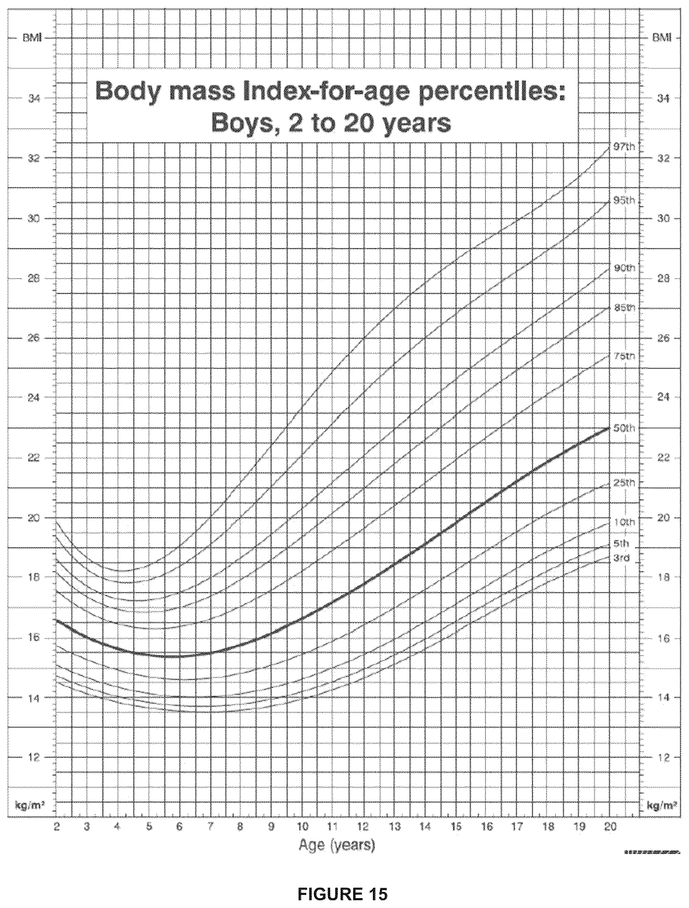

FIG. 16 shows Body Mass Index (BMI) for age percentiles for girls 2 to 20 years

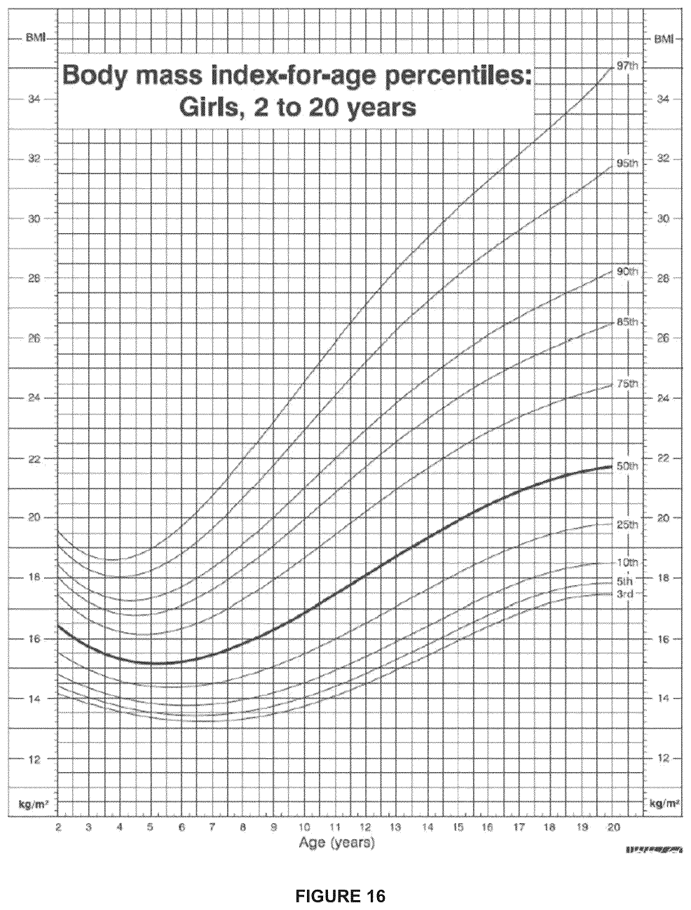

FIG. 17 Graphical study design

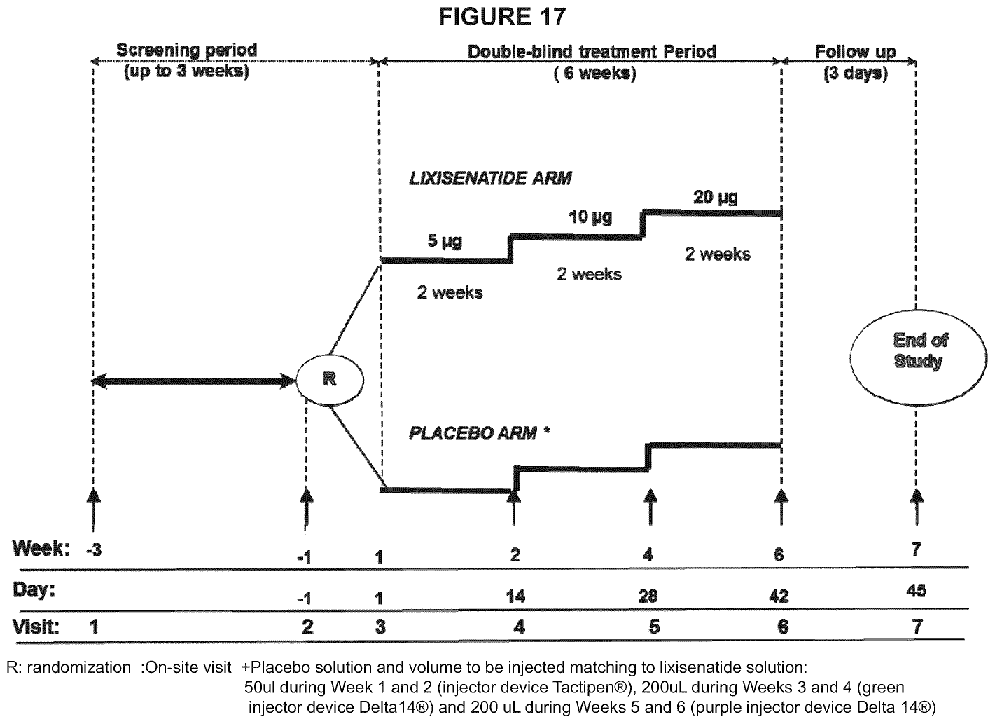

### SUBJECT-MATTER OF THE PRESENT APPLICATION IS DESCRIBED IN THE FOLLOWING ITEMS

1. Lixisenatide or/and a pharmaceutically acceptable salt thereof, for use in pediatrics.
2. Lixisenatide or/and a pharmaceutically acceptable salt thereof, for use according to item 1, wherein lixisenatide or/and the pharmaceutically acceptable salt thereof is administered as an add-on therapy to metformin or/and a pharmaceutically acceptable salt thereof.
3. Lixisenatide or/and a pharmaceutically acceptable salt thereof, for use according to item 2, wherein metformin or/and the pharmaceutically acceptable salt thereof is prepared for oral administration.
4. Lixisenatide or/and a pharmaceutically acceptable salt thereof, for use according to any one of the preceding items, wherein the patient in need of the pediatric treatment has an age of at least 10 years.
5. Lixisenatide or/and a pharmaceutically acceptable salt thereof, for use according to any one of the preceding items, wherein the patient in need of the pediatric treatment has an age of less than 18.
6. Lixisenatide or/and a pharmaceutically acceptable salt thereof, for use according to any one of the preceding items, wherein the patient in need of the pediatric treatment suffers from type 2 diabetes mellitus.
7. Lixisenatide or/and a pharmaceutically acceptable salt thereof, for use according to item 6, wherein the type 2 diabetes mellitus has been diagnosed at least three months before onset of therapy.
8. Lixisenatide or/and a pharmaceutically acceptable salt thereof, for use according to item 6 or 7, wherein the type 2 diabetes mellitus is not adequately controlled by metformin monotherapy, by basal insulin monotherapy or by a combination of metformin and a basal insulin.
9. Lixisenatide or/and a pharmaceutically acceptable salt thereof, for use according to any one of the preceding items, wherein the patient in need of the pediatric treatment is obese.
10. Lixisenatide or/and a pharmaceutically acceptable salt thereof, for use according to any one of the preceding items, wherein the patient in need of the pediatric treatment has a body mass index of at least 30 kg/m² or at least 31 kg/m².
11. Lixisenatide or/and a pharmaceutically acceptable salt thereof, for use according to any one of the preceding items, wherein lixisenatide is administered about 30 min before breakfast.
12. Lixisenatide or/and a pharmaceutically acceptable salt thereof, for use according to any one of the preceding items, wherein at the onset of treatment with lixisenatide or/and the pharmaceutically acceptable salt thereof, the patient has a fasting plasma glucose concentration of at least 8 mmol/L or at least 8.5 mmol/L.
13. Lixisenatide or/and a pharmaceutically acceptable salt thereof, for use according to any one of the preceding items, wherein at the onset of treatment with lixisenatide or/and the pharmaceutically acceptable salt thereof, the patient has a 2 hours postprandial plasma glucose concentration of at least 11.1 mmol/L or at least 12 mmol/L.
14. Lixisenatide or/and a pharmaceutically acceptable salt thereof, for use according to any one of the preceding items, wherein at the onset of treatment with lixisenatide or/and the pharmaceutically acceptable salt thereof, the patient has a glucose excursion of at least 3 mmol/L, wherein the glucose excursion is the difference of the 2 hours postprandial plasma glucose concentration and plasma glucose concentration 30 minutes prior to a meal test.
15. Lixisenatide or/and a pharmaceutically acceptable salt thereof, for use according to any one of the preceding items, wherein at the onset of treatment with lixisenatide or/and the pharmaceutically acceptable salt thereof, the patient has a HbA1c value of at least about 7%, at least about 7.5%, at least about 8%, at least about 8.5%, at least about 8.65%, or at least about 9%.
16. Lixisenatide or/and a pharmaceutically acceptable salt thereof, for use according to any one of the preceding items, wherein at the onset of treatment with lixisenatide or/and the pharmaceutically acceptable salt thereof, the patient has a plasma glucagon level of at least 140 ng/L.
17. Lixisenatide or/and a pharmaceutically acceptable salt thereof, for use according to any one of the preceding items, wherein at the onset of treatment with lixisenatide or/and the pharmaceutically acceptable salt thereof, the patient has a C-peptide plasma concentration of at least 1.2 nmol/L.
18. Lixisenatide or/and a pharmaceutically acceptable salt thereof, for use according to any one of the preceding items, wherein lixisenatide or/and the pharmaceutically acceptable salt thereof is prepared for parenteral administration.
19. Lixisenatide or/and a pharmaceutically acceptable salt thereof, for use according to any one of the preceding items, wherein lixisenatide is administered in a daily dose selected from the range of 5 μg to 10 μg.
20. Lixisenatide or/and a pharmaceutically acceptable salt thereof according to any one of the preceding items, for use in the improvement of glucose excursion, for use in the improvement of the postprandial plasma glucose concentration, or/and for use in the improvement of plasma glucagon concentration.
21. A pharmaceutical combination for use in pediatrics, said combination comprising
   (a) lixisenatide or/and a pharmaceutically acceptable salt thereof, and
   (b) metformin or/and a pharmaceutically acceptable salt thereof.
22. The pharmaceutical combination for use according to item 21, wherein lixisenatide or/and the pharmaceutically acceptable salt thereof is prepared for parenteral administration.
23. The pharmaceutical combination for use according to item 21 or 22, wherein metformin or/and the pharmaceutically acceptable salt thereof is prepared for oral administration.
24. The pharmaceutical combination for use according to any one of the item 21 to 23, wherein the patient in need of the pediatric treatment suffers from type 2 diabetes mellitus.
25. The pharmaceutical combination according to any one of the item 21 to 24, for use in the improvement of glucose excursion, for use in the improvement of the postprandial plasma glucose concentration, or/and for use in the improvement of plasma glucagon concentration.
26. A method of pediatric treatment, said method comprising administering to a patient in need of a pediatric treatment, lixisenatide or/and a pharmaceutically acceptable salt thereof.
27. The method according to item 26, wherein lixisenatide or/and the pharmaceutically acceptable salt thereof is prepared for parenteral administration.
28. The method according to item 26 or 27, further comprising administering metformin or/and a pharmaceutically acceptable salt thereof to the patient.
29. The method according to item 28, wherein metformin or/and the pharmaceutically acceptable salt thereof is prepared for oral administration.
30. The method according to any one of the items 26 to 29, wherein the patient in need of a pediatric treatment suffers from type 2 diabetes mellitus.
31. Use of lixisenatide or/and a pharmaceutically acceptable salt thereof, for the manufacture of a medicament for use in pediatrics.

1. A method of treating type 2 diabetes mellitus in a patient 10-17 years old comprising administering lixisenatide to the patient in need thereof at a dose of 5 μg, 10 μg or 20 μg per day,
wherein the treatment with lixisenatide reduces the levels of fasting plasma glucose (FPG), post-prandial glucose (PPG), and HbA_{1C} in the patient.

2. The method of claim 1, wherein the treatment with lixisenatide maintains or reduces body weight of the patient.

3. The method of claim 1, wherein the type 2 diabetes mellitus in the patient is not adequately controlled by oral anti-diabetic (“OAD”) treatment.

4. The method of claim 3, wherein the type 2 diabetes mellitus in the patient is not adequately controlled with metformin.

5. The method of claim 1, wherein the type 2 diabetes mellitus in the patient is not adequately controlled with basal insulin.

6. The method of claim 1, wherein the patient has a HbA1c>6.5% prior to treatment with lixisenatide.

7. The method of claim 4, wherein the lixisenatide is administered to the patient in need thereof as an add-on therapy to metformin.

8. The method of claim 5, wherein the lixisenatide is administered to the patient in need thereof as an add-on therapy to basal insulin.

9. The method of claim 1, wherein an initial dose of 5 μg is administered to the patient.

10. The method of claim 9, wherein the dose administered to the patient is increased from the initial dose 5 μg to a treatment dose of 20 μg.


SEQUENCE LISTING (2 sequences)

SEQ ID NO: 1 - protein, 44 aa, synthetic construct
  REGION 1..44: note = desPro36-Exendin-4(1-39)-Lys6-NH2
HGEGTFTSDLSKQMEEEAVRLFIEWLKNGGPSSGAPPSKKKKKK

SEQ ID NO: 2 - protein, 39 aa, Heloderma suspectum
HGEGTFTSDLSKQMEEEAVRLFIEWLKNGGPSSGAPPPS